(19) United States
(12) Patent Application Publication
(10) Pub. No.: US 2024/0382096 A1
(43) Pub. Date: Nov. 21, 2024

(54) MEDICAL INFORMATION PROCESSING APPARATUS, MEDICAL OBSERVATION SYSTEM, AND MEDICAL INFORMATION PROCESSING METHOD

(71) Applicant: SONY GROUP CORPORATION, TOKYO (JP)

(72) Inventors: SOTA SHOMAN, TOKYO (JP); SATOSHI OZAKI, TOKYO (JP); SHO INAYOSHI, TOKYO (JP); YUGO KATSUKI, TOKYO (JP); HIROSHI YOSHIDA, TOKYO (JP)

(21) Appl. No.: 18/556,070

(22) PCT Filed: Feb. 14, 2022
(86) PCT No.: PCT/JP2022/005556

§ 371 Date: Oct. 18, 2023

(30) Foreign Application Priority Data
May 10, 2021 (JP) 2021-079780

(51) Int. Cl. A61B 5/02; A61B 1/00; A61B 1/06; A61B 1/313; A61B 5/00; H04N 23/74; H04N 25/47

(52) U.S. Cl. CPC A61B 5/02042; A61B 1/0005; A61B 1/0638; A61B 1/0655; A61B 1/3132; A61B 5/7282; H04N 23/74; H04N 25/47

(57) ABSTRACT

A processing apparatus (40), which is an example of a medical information processing apparatus according to an aspect of the present disclosure, includes a signal processing unit (41), which is an example of a bleeding detection unit configured to detect a bleeding portion in a body based on a detection result of an EVS (21), which is an example of an event detection unit including a plurality of first pixels, each of the first pixels receiving light reflected in the body irradiated by a light source (30), and detecting, for each of the first pixels, that a luminance change amount due to the light incident on the first pixel exceeds a predetermined threshold value depending on a change in a wavelength or an irradiation direction of the light source (30).

### FIELD

The present disclosure relates to a medical information processing apparatus, a medical observation system, and a medical information processing method.

### BACKGROUND

In surgery such as laparoscopic surgery, a worker such as an operator or an assistant is less likely to notice a bleeding portion, for example, a bleeding portion at a position that is difficult to see or a bleeding portion outside a region of interest due to a color of an organ inside a body of a patient. If a response to bleeding in the body of the patient is delayed during surgery, a burden on the patient increases.

Meanwhile, a detection technique for detecting a liquid or a foreign substance in the liquid has been developed. For example, as a technique for detecting a foreign substance in a liquid, proposed is a foreign substance detection technique for detecting a foreign substance in a liquid contained in a container (for example, refer to Patent Literature 1). In this foreign substance detection technique, a detection target is not a liquid but a foreign substance, and the foreign substance is detected using illumination and specular reflection thereof.

### CITATION LIST

### Patent Literature

Patent Literature 1: JP 2008-309806 A

### SUMMARY

### Technical Problem

However, in the foreign substance detection technique described above, since the liquid needs to be present in the container, it is difficult to apply the foreign substance detection technique to a surgical environment such as laparoscopic surgery, and it is difficult to detect a bleeding portion in the body. Therefore, it is required to enable detection of a bleeding portion in the body in a surgical environment such as laparoscopic surgery, that is, in an environment in which the body of a patient or the like is operated.

Therefore, the present disclosure proposes a medical information processing apparatus, a medical observation system, and a medical information processing method capable of detecting a bleeding portion in the body.

### Solution to Problem

A medical information processing apparatus according to the embodiment of the present disclosure includes: a bleeding detection unit configured to detect a bleeding portion in a body based on a detection result of an event detection unit including a plurality of first pixels, each of the first pixels receiving light reflected in the body irradiated by a light source, the event detection unit detecting, for each of the first pixels, that a luminance change amount due to the light incident on the first pixel exceeds a predetermined threshold value depending on a change in a wavelength or an irradiation direction of the light source.

A medical observation system according to the embodiment of the present disclosure includes: a light source; an event detection unit including a plurality of first pixels, each of the first pixels receiving light reflected in a body irradiated by the light source, the event detection unit being configured to detect, for each of the first pixels, that a luminance change amount due to the light incident on the first pixel exceeds a predetermined threshold value depending on a change in a wavelength or an irradiation direction of the light source; and a bleeding detection unit configured to detect a bleeding portion in the body based on a detection result of the event detection unit.

A medical information processing method according to the embodiment of the present disclosure includes: by a medical information processing apparatus, detecting a bleeding portion in a body based on a detection result of an event detection unit including a plurality of first pixels, each of the first pixels receiving light reflected in the body irradiated by a light source, the event detection unit detecting, for each of the first pixels, that a luminance change amount due to the light incident on the first pixel exceeds a predetermined threshold value depending on a change in a wavelength or an irradiation direction of the light source.

### BRIEF DESCRIPTION OF DRAWINGS

FIG. 1 is a diagram illustrating an example of a schematic configuration of a medical observation device according to a first embodiment.

FIG. 2 is a diagram illustrating an example of a schematic configuration of a camera according to the first embodiment.

FIG. 3 is a diagram illustrating an example of a schematic configuration of an EVS according to the first embodiment.

FIG. 4 is a diagram illustrating an example of a schematic configuration of a pixel according to the first embodiment.

FIG. 5 is a diagram illustrating an example of bleeding detection processing by a wavelength change in irradiation light according to the first embodiment.

FIG. 6 is a graph illustrating a relationship between a wavelength and an intensity of absorption/scattering according to the first embodiment.

FIG. 7 is a flowchart illustrating an example of a flow of the bleeding detection processing by the wavelength change in irradiation light according to the first embodiment.

FIG. 8 is a first diagram illustrating an example of display of a bleeding detection result according to the first embodiment.

FIG. 9 is a second diagram illustrating an example of the display of the bleeding detection result according to the first embodiment.

FIG. 10 is a third diagram illustrating an example of the display of the bleeding detection result according to the first embodiment.

FIG. 11 is a diagram illustrating an example of bleeding detection processing by a change in irradiation direction of irradiation light according to a second embodiment.

FIG. 12 is a diagram illustrating the change in irradiation direction of the irradiation light according to the second embodiment.

FIG. 13 is a flowchart illustrating an example of a flow of the bleeding detection processing by the change in irradiation direction of the irradiation light according to the second embodiment.

FIG. 14 is a diagram illustrating an example of a schematic configuration of an endoscope system.

FIG. 15 is a block diagram illustrating an example of a functional configuration of a camera and a camera control unit (CCU) illustrated in FIG. 14.

FIG. 16 is a diagram illustrating an example of a schematic configuration of a microscopic surgery system.

### DESCRIPTION OF EMBODIMENTS

Hereinafter, embodiments of the present disclosure will be described in detail with reference to the accompanying drawings. It is noted that, in the present specification and the drawings, components having substantially the same functional configuration are basically denoted by the same reference numerals, and a redundant description is omitted. An apparatus, a method, a system, and the like according to the present disclosure are not limited by the embodiments of the present disclosure.

Further, one or more embodiments (including examples and modifications) described below can each be implemented independently of each other. On the other hand, at least some of the plurality of embodiments described below may be appropriately combined with at least some of other embodiments to be implemented. The plurality of embodiments may include novel features different from each other. Therefore, the plurality of embodiments can contribute to solving different objects or problems, and can exhibit different effects. It is noted that an apparatus, a method, a system, and the like according to the present disclosure are not limited by one or more embodiments.

The present disclosure will be described according to the following order of items.

### 1. First Embodiment

1-1. Configuration example of medical observation system
1-2. Configuration example of camera
1-3. Configuration example of EVS
1-4. Configuration example of pixel
1-5. Processing example of bleeding detection
1-6. Display example of bleeding detection result
1-7. Action and effect
2. Second embodiment
2-1. Processing example of bleeding detection
2-2. Action and effect
3. Other embodiments
4. Application example
5. Appendix

### 1. First Embodiment

### <1-1. Configuration Example of Medical Observation System>

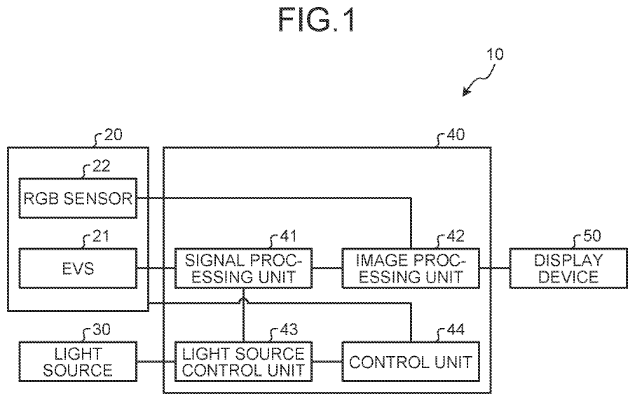

A configuration example of a medical observation system 10 according to the present embodiment will be described with reference to FIG. 1. FIG. 1 is a diagram illustrating an example of a schematic configuration of the medical observation system 10 according to the present embodiment.

As illustrated in FIG. 1, the medical observation system 10 includes a camera (imaging device) 20, a light source (light source device) 30, a processing apparatus 40, and a display device 50. The processing apparatus 40 corresponds to a medical information processing apparatus.

The camera 20 includes an event-based vision sensor (EVS) 21 and an RGB sensor 22. The EVS 21 corresponds to an event detection unit. Furthermore, the RGB sensor 22 corresponds to an image detection unit.

The EVS 21 mainly includes, for example, a plurality of pixels (first pixels) arranged in a matrix, and a peripheral circuit unit that outputs an event or an image based on light incident on each of the plurality of pixels as an event signal (event data) and a pixel signal (both are not illustrated). The event signal and the pixel signal output from the EVS 21 are transmitted to the processing apparatus 40.

Specifically, each pixel of the EVS 21 detects the presence or absence of an event by comparing an amount of change in photocurrent corresponding to a change in luminance of the incident light with a predetermined threshold value. For example, the pixel detects, as an event, a fact that a luminance change amount exceeds a predetermined threshold value. The EVS 21 will be described below in detail.

The RGB sensor 22 mainly includes, for example, a plurality of pixels (second pixels) arranged in a matrix, and a peripheral circuit unit that outputs an image based on light incident on each of the plurality of pixels as a pixel signal (both are not illustrated). The pixel signal output from the RGB sensor 22 is transmitted to the processing apparatus 40.

Specifically, the RGB sensor 22 is, for example, a color photographable image sensor having a Bayer array capable of detecting blue light, green light, and red light. Furthermore, the RGB sensor 22 is preferably, for example, an image sensor capable of coping with photographing of a high resolution image of 4K or more. By using such an image sensor, an image of a surgical portion can be obtained with high resolution, and as such an operator such as an operating surgeon can grasp the state of the surgical portion in more detail, and can smoothly proceed the surgery.

It is noted that the RGB sensor 22 may include a pair of image sensors for acquiring right-eye and left-eye images corresponding to 3D display (stereo system). By performing the 3D display, an operator such as an operating surgeon can more accurately grasp the depth of a living tissue (organ) in the surgical portion and can grasp a distance to the living tissue.

The light source 30 irradiates an object to be imaged with light. The light source 30 is a light source capable of continuously or stepwise changing a wavelength and an irradiation direction of irradiation light. The light source 30 may be implemented by, for example, a plurality of light emitting diodes (LEDs) having different wavelengths. In this case, by finely adjusting each LED individually, it is possible to reproduce various types of light having various spectral distributions and to change the wavelength. Furthermore, the light source 30 may be implemented by, for example, combining one or a plurality of LEDs and a lens, or combining one or a plurality of optical fibers. In this case, light from the LED or the optical fiber is scanned by the lens, and the light irradiation direction can be changed.

It is noted that, in a case where only the wavelength of the irradiation light is changed, that is, in a case where the light from the light source 30 may be diffused, the light source 30 may be implemented by, for example, an LED for a wide-angle lens. Furthermore, for example, the light source 30 may be configured to diffuse light by combining a normal LED and a lens. Furthermore, for example, the light source 30 may be configured to diffuse light transmitted by an optical fiber (light guide) with a lens. In addition, the light source 30 may expand an irradiation range by directing the optical fiber itself in a plurality of directions to perform light irradiation.

The processing apparatus 40 includes a signal processing unit 41, an image processing unit 42, a light source control unit 43, and a control unit 44. The processing apparatus 40 includes, for example, a computer such as a central processing unit (CPU) or a micro control unit (GPU), and can integrally control operations of the camera 20, the light source 30, the display device 50, and the like.

The signal processing unit 41 can perform various types of processing on the event data and the pixel signal obtained from the EVS 21. Furthermore, the signal processing unit 41 provides information obtained by the processing to the image processing unit 42. For example, the signal processing unit 41 detects a bleeding portion in a body of a living body based on the event data and transmits the detected bleeding portion to the image processing unit 42. The detection of the bleeding portion will be described below in detail. The signal processing unit 41 corresponds to a bleeding detection unit.

The image processing unit 42 can perform various types of image processing for displaying an image with respect to a pixel signal received from the EVS 21 or the RGB sensor 22. Furthermore, the image processing unit 42 provides the pixel signal subjected to the image processing to the display device 50. For example, the image processing unit 42 processes the bleeding portion obtained by the signal processing unit 41 and an image obtained from the RGB sensor 22, and generates an image in which the bleeding portion is emphasized. Generation of the image in which the bleeding portion is emphasized will be described below in detail.

The light source control unit 43 controls the light source 30 so as to change a wavelength or an irradiation direction (a wavelength or an irradiation direction of light emitted from the light source 30) of the light source 30. For example, the light source control unit 43 can transmit a control signal to the light source 30 to control driving thereof. The control signal for the light source 30 may include information regarding irradiation conditions such as a wavelength and an irradiation direction of irradiation light.

The control unit 44 controls the camera 20 (the EVS 21 and the RGB sensor 22), the light source control unit 43, and the like. For example, the control unit 44 can transmit a control signal to each of the EVS 21, the RGB sensor 22, and the light source control unit 43 so as to control driving thereof. The control signals for the EVS 21 and the RGB sensor 22 may include information regarding imaging conditions such as a magnification and a focal length.

The display device 50 displays various images. The display device 50 displays, for example, an image captured by the camera 20 (the EVS 21 and the RGB sensor 22). The display device 50 is implemented by, for example, a display including a liquid crystal display (LCD), an organic electro-luminescence (EL) display, or the like. It is noted that the display device 50 may be a device integrated with the processing apparatus 40, or may be a separate device connected to the processing apparatus 40 so as to be able to communicate therewith in a wired or wireless manner.

It is noted that the various images may be stored in a storage unit (not illustrated) or the like as necessary. The storage unit is implemented by, for example, a storage such as a flash memory, a dynamic random access memory (DRAM), or a static random access memory (SRAM).

### <1-2. Configuration Example of Camera>

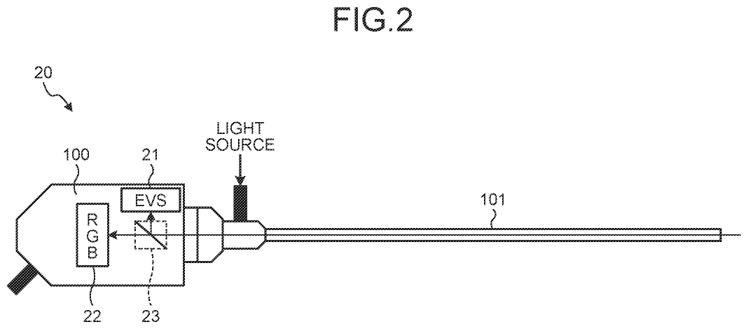

A configuration example of the camera 20 according to the present embodiment will be described with reference to FIG. 2. FIG. 2 is a diagram illustrating an example of the schematic configuration of the camera 20 according to the present embodiment.

As illustrated in FIG. 2, the camera 20 includes a beam splitter 23, a camera head 100, and an optical system 101 in addition to the EVS 21 and the RGB sensor 22. The beam splitter 23 corresponds to a spectroscopic unit.

The camera head 100 incorporates the RGB sensor 22, the EVS 21, and the beam splitter 23. The beam splitter 23 is a member configured to divide light reflected by a subject and to guide the reflected light to both the EVS 21 and the RGB sensor 22. The beam splitter 23 is implemented by, for example, a prism. The beam splitter 23 functions as, for example, a half mirror.

The optical system 101 guides light from the light source 30 to the subject, and guides the light reflected by the subject to the camera 20. The optical system 101 includes a light source optical system and an imaging optical system (both are not illustrated), guides the light from the light source 30 to the subject by the light source optical system, and further guides the light reflected by the subject to the camera 20 by the imaging optical system. For example, the optical system 101 is configured by combining a plurality of lenses including a zoom lens and a focus lens. The zoom lens and the focus lens may be configured to be movable in position on the optical axis for adjustment of magnification and focus of a captured image.

For example, such a camera 20 can capture images of various objects to be imaged (for example, an intraperitoneal environment). Furthermore, the camera 20 can capture, for example, a surgical field image including various surgical tools, organs, and the like in the abdominal cavity of a patient. Specifically, the camera 20 functions as an imaging unit capable of capturing an image of a capturing target in the form of a moving image or a still image. Furthermore, the camera 20 can transmit an electric signal (a pixel signal) corresponding to the captured image to the processing apparatus 40.

Here, in the present embodiment, by providing the camera 20 in combination with the EVS 21 and the RGB sensor 22, it is possible to utilize both the high dynamic range, the high robustness of fast moving subject detection, and the high time resolution, which are features of the EVS 21, and the high tracking performance for a long time, which is a feature of the RGB sensor 22, and as such it is possible to improve recognition accuracy of a subject.

It is noted that, in the present embodiment, the camera 20 may be, for example, an oblique endoscope, a front direct endoscope with a wide angle/cutout function, an endoscope with a distal end bending function, or an endoscope with a simultaneous photographing function in another direction, or may be an external endoscope or a microscope, and is not particularly limited. Furthermore, the camera 20 may be a stereoscopic endoscope capable of measuring a distance. Alternatively, a depth sensor (a distance measuring device) may be provided in the camera head 100 or separately from the camera head 100. The depth sensor is, for example, a sensor that performs distance measurement using a time of flight (ToF) method of performing distance measurement using a return time of reflection of pulsed light from a subject or a structured light method of performing distance measurement by distortion of a pattern by emitting grid-like pattern light.

Furthermore, in the present embodiment, configurations of the camera head 100, the optical system 101, and the like may be based on a sealed structure having high airtightness and waterproofness. As a result, the camera head 100 and the optical system 101 can have resistance to autoclave sterilization.

Furthermore, in the present embodiment, for example, the camera head 100 and the optical system 101 may be provided at the distal end of a robot arm. The robot arm supports the camera head 100 and the optical system 101. It is noted that the robot arm may support a surgical tool such as forceps.

Furthermore, in the present embodiment, it has been described that the EVS 21 and the RGB sensor 22 are mounted on the same camera head 100, but may be respectively mounted on two different camera heads. Furthermore, in a case where a camera head having the EVS 21 mounted thereon and a camera head having the RGB sensor 22 mounted thereon are different from each other, the camera heads may be respectively supported by different robot arms.

Furthermore, in the present embodiment, it has been described that light is guided to both the EVS 21 and the RGB sensor 22 by the beam splitter 23, but the present disclosure is not limited thereto. For example, a hybrid-type sensor in which pixel arrays corresponding to the EVS 21 and the RGB sensor 22 are provided on the same substrate (light receiving surface) may be used. In such a case, since the beam splitter 23 is unnecessary, the configuration in the camera head 100 can be simplified.

Furthermore, in the present embodiment, the beam splitter 23 may have a function of adjusting a distribution ratio of the light amount of light incident on each of the EVS 21 and the RGB sensor 22. For example, the above function can be provided by adjusting transmittance of the beam splitter 23. More specifically, for example, in a case where the optical axis of the incident light is the same between the EVS 21 and the RGB sensor 22, it is preferable to adjust the transmittance of the beam splitter 23 so as to increase the light amount of light incident on the side of the RGB sensor 22.

Furthermore, in the present embodiment, each of the EVS 21 and the RGB sensor 22 may be provided with two or three or more in order to enable a stereo system capable of performing distance measurement. Furthermore, in a case where the stereo system is to be implemented, two image circles may be projected on one pixel array by causing two optical systems to correspond to one pixel array.

Furthermore, in the present embodiment, the EVS 21 and the RGB sensor 22 may be provided in the distal end portion of a flexible endoscope or a rigid endoscope inserted into the abdominal cavity.

### <1-3. Configuration Example of EVS>

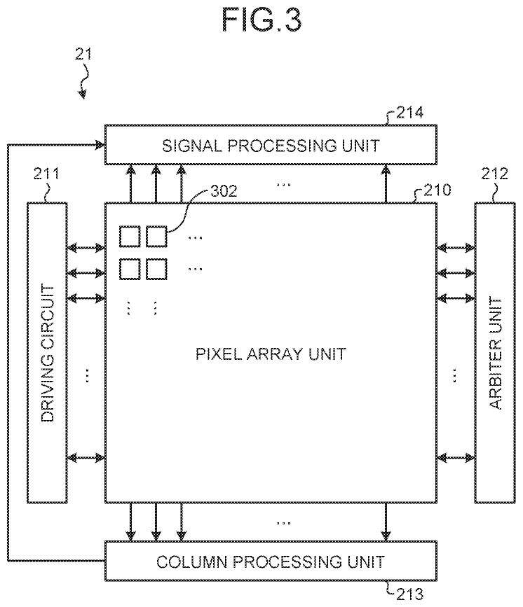

A configuration example of the EVS 21 according to the present embodiment will be described with reference to FIG. 3. FIG. 3 is a diagram illustrating an example of a schematic configuration of the EVS 21 according to the present embodiment.

As illustrated in FIG. 3, the EVS 21 includes a pixel array unit 210, a driving circuit (driving unit) 211, an arbiter unit (arbitration unit) 212, a column processing unit 213, and a signal processing unit 214. The driving circuit 211, the arbiter unit 212, the column processing unit 213, and the signal processing unit 214 are provided as peripheral circuit units of the pixel array unit 210.

The pixel array unit 210 includes a plurality of pixels (first pixels) 302. These pixels 302 are arranged in a matrix (for example, a matrix shape). Each of the pixels 302 generates a voltage corresponding to a photocurrent generated by photoelectric conversion as a pixel signal. Furthermore, each pixel 302 detects presence or absence of an event by comparing a change in photocurrent corresponding to a change in luminance of incident light with a predetermined threshold value. For example, each pixel 302 detects an event based on a luminance change amount exceeding a predetermined threshold value.

When detecting the event, each pixel 302 outputs, to the arbiter unit 212, a request for requesting the output of event data indicating occurrence of the event. Then, when receiving a response indicating permission for the output of the event data from the arbiter unit 212, each pixel 302 outputs the event data to the driving circuit 211 and the signal processing unit 214. Furthermore, the pixel 302 that has detected the event outputs a pixel signal generated by photoelectric conversion to the column processing unit 213.

The driving circuit 211 can drive each pixel 302 of the pixel array unit 210. For example, the driving circuit 211 drives the pixel 302 that has detected an event and output event data, and outputs a pixel signal of the corresponding pixel 302 to the column processing unit 213.

The arbiter unit 212 arbitrates a request for requesting the output of the event data supplied from each of the pixels 302, and transmits a response based on an arbitration result (permission/non-permission of the output of the event data) and a reset signal for resetting event detection to the pixel 302.

For each column of the pixel array unit 300, the column processing unit 213 performs processing of converting an analog pixel signal output from the pixel 302 in the corresponding column into a digital signal. For example, the column processing unit 213 performs correlated double sampling (CDS) processing on a digitized pixel signal.

The signal processing unit 214 performs predetermined signal processing on the digitized pixel signal supplied from the column processing unit 213 and the event data output from the pixel array unit 210, and outputs the event data (time stamp information and the like) and the pixel signal after the signal processing.

Here, a change in photocurrent generated in the pixel 302 can be regarded as a light amount change (a luminance change) of light incident on the pixel 302. Therefore, it can also be said that an event is a luminance change) of the pixel 302 exceeding a predetermined threshold value. Furthermore, the event data indicating occurrence of the event can include at least position information such as coordinates indicating the position of the pixel 302 in which a light amount change as an event has occurred. The event data can include polarity of a light amount change in addition to the position information.

### <1-4. Configuration Example of Pixel>

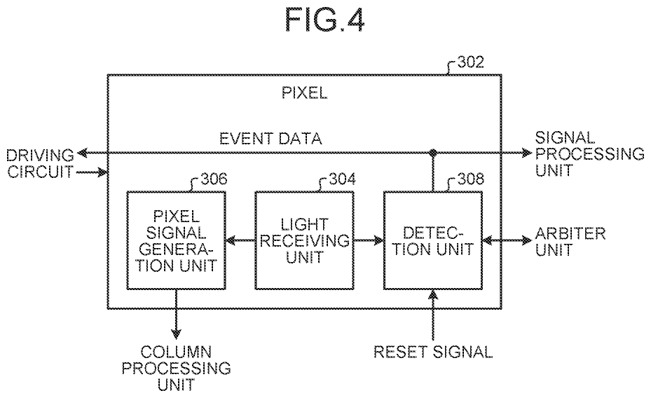

A configuration example of the pixel 302 according to the present embodiment will be described with reference to FIG. 4. FIG. 4 is a diagram illustrating an example of a schematic configuration of the pixel 302 according to the present embodiment.

As illustrated in FIG. 4, each pixel 302 includes a light receiving unit 304, a pixel signal generation unit 306, and a detection unit 308.

The light receiving unit 304 photoelectrically converts incident light to generate a photocurrent. Then, the light receiving unit 304 supplies a signal of a voltage corresponding to the photocurrent to either the pixel signal generation unit 306 or the detection unit 308 under the control of the driving circuit 211 (refer to FIG. 3). As the light receiving unit 304, for example, a photoelectric conversion element such as a photodiode is used.

The pixel signal generation unit 306 generates the signal supplied from the light receiving unit 304 as a pixel signal. Then, the pixel signal generation unit 306 supplies the generated analog pixel signal to the column processing unit 213 via a vertical signal line VSL (not illustrated) corresponding to the column of the pixel array unit 210.

The detection unit 308 detects whether or not an event has occurred based on whether or not a change amount of the photocurrent from the light receiving unit 304 exceeds a predetermined threshold value. The event includes, for example, an on-event indicating that the change amount of the photocurrent exceeds an upper limit threshold value and an off-event indicating that the change amount falls below a lower limit threshold value. It is noted that the detection unit 308 may detect only the on-event.

When the event occurs, the detection unit 308 outputs a request for requesting an output of event data indicating occurrence of the event to the arbiter unit 212. Then, when receiving a response to the request from the arbiter unit 212, the detection unit 308 outputs the event data to the driving circuit 211 and the signal processing unit 214.

It is noted that, in the present embodiment, the pixel signal generation unit 306 and the detection unit 308 are provided for each of the pixels 302, but the present disclosure is not limited thereto, and for example, the pixel signal generation unit 306 and the detection unit 308 may be provided in common for each of the plurality of pixels 302. For example, four pixels 302 may be treated as one pixel (block pixel), and the pixel signal generation unit 306 and the detection unit 308 may be provided for each block pixel.

By applying such an EVS 21 to the medical observation system 10, it is possible to utilize a high dynamic range, high robustness of fast moving subject detection, and high temporal resolution which are characteristics of the EVS 21, and as such it is possible to improve recognition accuracy of a subject.

Here, the EVS 21 is an image sensor that sensitively detects a luminance change, and usually has higher sensitivity than that of the RGB sensor 22. Therefore, the shape (edge information) of a subject can be easily obtained even in a dark place that is difficult to be captured by the RGB sensor 22.

Furthermore, the EVS 21 can sparsely output time stamp information and pixel information when the luminance change exceeds a threshold value without depending on a frame rate. Therefore, the EVS 21 can perform an output at a high frame rate depending on a frequent luminance change. Furthermore, by integrating the output from the EVS 21 for a certain period and converting the same as an image with a suitable frame rate, movement and deformation of a subject can be easily captured. Specifically, since the number of events to be integrated changes depending on a frame length, information included in the converted image also changes. Therefore, by performing the processing of adjusting the frame length in order to capture a desired subject and the deformation thereof using the features of the EVS 21, the shape (edge information) of the subject can be more easily obtained, and recognition performance of the subject can be improved.

In addition, since the EVS 21 does not output all the pixel information for each constant frame, the amount of data is usually smaller than that of the RGB sensor 22, and arithmetic processing can be performed with a lighter load.

### <1-5. Processing Example of Bleeding Detection>

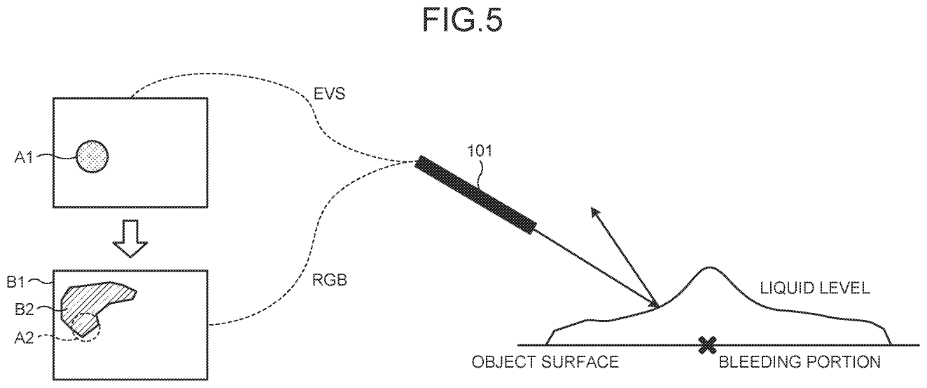

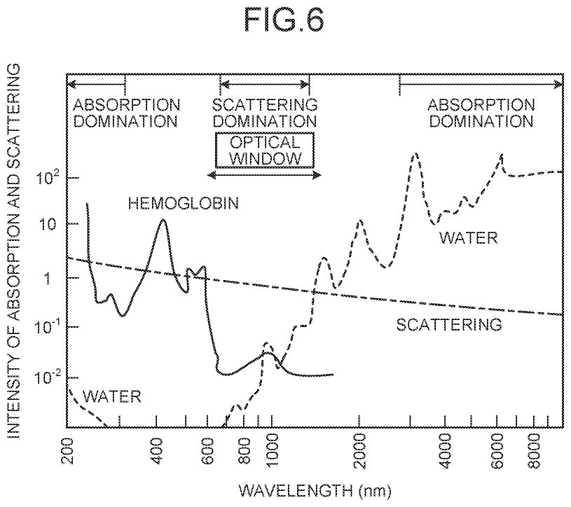

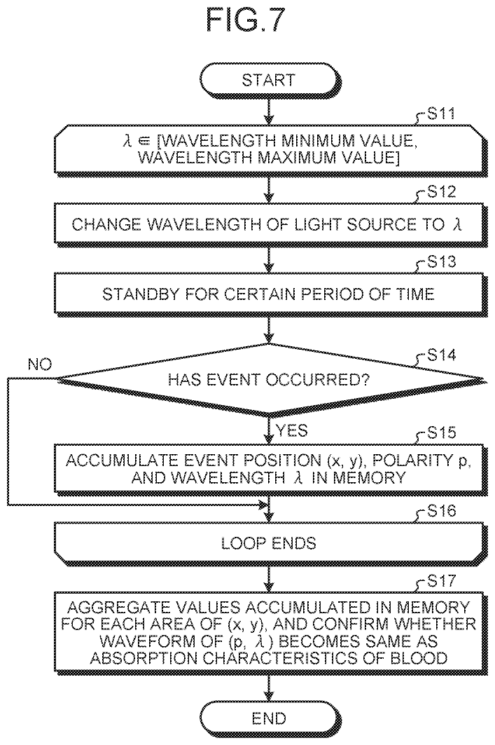

A processing example of bleeding detection by a wavelength change of irradiation light according to the present embodiment will be described with reference to FIGS. 5 to 7. FIG. 5 is a diagram illustrating an example of bleeding detection processing by a wavelength change of irradiation light according to the present embodiment. FIG. 6 is a graph illustrating a relationship between a wavelength and an intensity of absorption/scattering according to the present embodiment. FIG. 7 is a flowchart illustrating an example of a flow of the bleeding detection processing by the wavelength change of the irradiation light according to the present embodiment.

As illustrated in FIG. 5, irradiation light guided by the optical system 101 travels toward an object surface corresponding to a surface of a body (for example, intraperitoneal) of a living body, and the irradiation light is reflected by the object surface or a liquid level of a bleeding portion. A part of the reflected light (reflected light) is incident on the optical system 101. The light (incident light) incident on the optical system 101 is guided to the camera 20 (refer to FIG. 2) by the optical system 101. In the camera 20, an event is detected by the EVS 21 depending on a luminance change based on the incident light, and an image based on the incident light is obtained by the RGB sensor 22. Thereafter, a bleeding portion A1, which is the bleeding portion, is obtained by the signal processing unit 41 (refer to FIG. 1) based on a detection result of the event. Then, a figure (for example, a circle or the like) A2 indicating the bleeding portion A1 is superimposed on an image B1, and a display image is generated by the image processing unit 42 (refer to FIG. 1). In the example of FIG. 5, an organ B2 is displayed in the image B1, and the figure A2 is superimposed on a part of the organ B2. The figure A2 indicates a position and a region of the bleeding portion A1.

Here, as illustrated in FIG. 6, it is known that light absorption characteristics vary depending on a substance hit by light. For example, when a wavelength of the light source 30 is changed, an intensity of reflected light changes depending on light absorption characteristics of an object hit by light. A material of the object can be detected by detecting how the reflected light changes. When blood containing hemoglobin easily absorbs a wavelength by changing the wavelength of the irradiation light (the wavelength of the light source 30) within a predetermined range of 400 to 800 nm, for example, reflected light by the blood becomes weak and a luminance value of the EVS 21 becomes low. On the other hand, when blood hardly absorbs the wavelength, the reflected light by the blood becomes strong, and the luminance value of the EVS 21 becomes high. By using such an EVS 21, it is possible to detect only the luminance change accompanying the wavelength change of the light source 30 with high speed and high sensitivity, and it is possible to reliably detect a bleeding portion.

In the example of FIG. 6, water reflection characteristics are also illustrated. That is, it is possible to distinguish between water and blood (hemoglobin). In addition to water, reflection characteristics of an object such as an organ may be used. In this case, it is possible to distinguish between an object such as an organ and blood. Therefore, a bleeding portion can be detected more accurately.

As illustrated in FIG. 7, as bleeding detection processing, in step S11, a wavelength λ of the light source 30 is changed from a minimum value (a wavelength minimum value) to a maximum value (a wavelength maximum value). In the example of FIG. 7, steps S11 to S16 are performed in a loop, and the processing in the loop is repeatedly performed until the wavelength λ of the light source 30 is changed from the minimum value to the maximum value. An increase amount in the wavelength for each loop is set to a predetermined value such as 1 or 10 μm. Further, a predetermined range of a wavelength change is, for example, 400 to 800 nm.

In step S12, the wavelength λ of the light source 30 is changed by the light source control unit 43, and a standby state for a certain period of time is executed in step S13. The certain period of time is, for example, 0.01 seconds or the like, and is a period of time for event detection. An event is detected pixel by pixel by the EVS 21. It is noted that a threshold value related to occurrence of an event is set to a value with which a bleeding portion can be detected.

Next, in step S14, the EVS 21 determines whether or not an event has occurred. When it is determined that the event has occurred (Yes in step S14), an event position (x, y), a polarity p, and a wavelength λ are associated with each other and accumulated in a memory in step S15. The event position is a position of a pixel in which an event has occurred (the event has been detected) in an XY coordinate system. Further, the polarity p indicates a luminance polarity, and is information on a luminance change amount (for example, a luminance difference or the like). It is noted that the memory is provided in the signal processing unit 41, but is not limited thereto. For example, the memory may be provided in the EVS 21.

After the processing in step S15 or when it is determined in step S14 that no event has occurred (No in step S14), the processing proceeds to step S16. When the loop ends in step S16 (when the wavelength λ reaches the maximum value), the values accumulated in the memory are aggregated for each area of the event position (x, y) in step S17, and it is confirmed whether a waveform (for example, a waveform of the luminance change amount depending on the wavelength change) of (p, λ) becomes the same as absorption characteristics of blood. The aggregation processing and the confirmation processing are executed by the signal processing unit 41. It is noted that the absorption characteristics of blood are stored, for example, by the signal processing unit 41. For example, when the waveform of (p, λ) becomes the same as the absorption characteristics of blood, it is determined that bleeding has occurred. In this case, an alert notifying an operator or the like that there is bleeding may be issued.

According to such bleeding detection processing, the wavelength of the light source 30 is changed from the minimum value to the maximum value, and the events occurring during that time are accumulated in the memory. By aggregating events occurring in a close range on the image, a luminance change with respect to the wavelength is observed. Among the luminance changes, if there is a portion having a luminance change similar to a luminance change with respect to blood, it can be understood that blood exists at a position of the portion, and bleeding can be notified. By using the EVS 21 capable of detecting the luminance change at high speed and high sensitivity, the time required for the wavelength change of the light source 30 can be shortened, thereby making it possible to implement faster bleeding detection.

### <1-6. Display Example of Bleeding Detection Result>

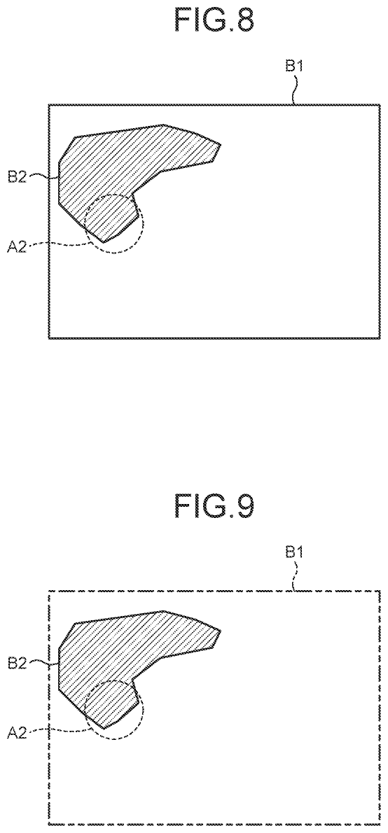

An example of display of a bleeding detection result according to the present embodiment will be described with reference to FIGS. 8 to 10. FIGS. 8 to 10 are diagrams each illustrating an example of display of a bleeding detection result according to the present embodiment.

As illustrated in FIG. 8, an image B1 including an annular figure A2 and an organ B2 is displayed by the display device 50. In the example of FIG. 8, the figure A2 is a dotted circle. The figure A2 is superimposed on a part of the organ B2 in the image B1 and indicates a bleeding portion. As a result, an operator such as an operating surgeon can visually recognize the figure A2 in the image B1 and accurately grasp the bleeding portion, thereby making it possible to quickly respond to bleeding in the patient's body.

As the figure A2, for example, various shapes and line types can be used in addition to the circular dotted line. The shape of the figure A2 may be, for example, a geometric shape such as a square or a triangle, or a free shape, or may be a shape pointed by an arrow or the like. However, the shape of the figure A2 is preferably a shape indicating the region of the bleeding portion A1. In addition, in a case where it is difficult for the operator to see a surgical portion due to the figure A2, the figure A2 may be blinked.

As illustrated in FIG. 9, basically the same display as that in FIG. 8 is performed. However, in FIG. 9, in order to make it easy for an operator to understand that there is bleeding in the body, a color, a line type, and the like of a frame line of the image B1 can be changed as compared with FIG. 8. At this time, both or one of the color and the line type is changed. For example, the frame line is black when there is no bleeding and turns red when there is bleeding. In addition, the frame line is a solid line when there is no bleeding, and is changed to a dotted line when there is bleeding. As a result, an operator such as an operating surgeon can quickly and reliably grasp that there is bleeding, thereby making it possible to quickly respond to bleeding in the patient's body.

It is noted that a thickness of the frame line may be changed in addition to the color and the line type of the frame line depending on the presence or absence of bleeding in the body. In addition, in order to make it easier for the operator to understand that there is bleeding, the frame line may be blinked, or the image B1 may be blinked for a certain time such as several seconds.

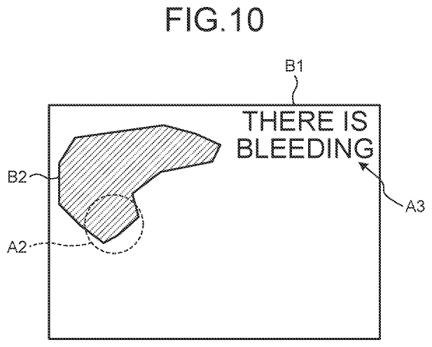

As illustrated in FIG. 10, basically the same display as that in FIG. 8 is performed. However, in FIG. 10, in order to make it easy for an operator to understand that there is bleeding, a text A3 indicating “there is bleeding” is displayed to be superimposed on the image B1. As a result, an operator such as an operating surgeon can quickly and reliably grasp that there is bleeding, thereby making it possible to quickly respond to bleeding in the patient's body.

It is noted that a display position of the text A3 is on the upper right of the image B1 in the example of FIG. 10, but the display position is not limited thereto, and may be at the center of the image B1 in order to make it easier for an operator to understand that there is bleeding. At this time, the text A3 may be blinked in order to suppress the operator from having difficulty in seeing a surgical portion.

According to such a display example of the bleeding detection result, the display image is an image in which the figure A2 having a geometric shape such as a circle indicating a place at which bleeding is detected is superimposed on the image B1 captured by the RGB sensor 22. In addition, in order to make it easy to understand that there is bleeding, a color of a frame line of the image B1 may be changed, or a warning may be issued by text display. By performing display so as to recognize where bleeding is occurring, an operator such as an operating surgeon can immediately perform a treatment corresponding to bleeding, and a burden on the patient can be reduced.

Here, as described above, the display device 50 functions as a notification unit configured to notify that there is a bleeding portion in the body of a living body such as a patient, but the present disclosure is not limited thereto. For example, as the notification unit, for example, a warning light such as a lamp or a sound output unit such as a speaker may be used in addition to the display device 50. The sound output unit can notify that there is a bleeding portion in the body of a living body by, for example, a warning sound, voice, music, or the like.

### <1-7. Action and Effect>

As described above, according to the first embodiment, there is provided a bleeding detection unit (for example, the signal processing unit 41) configured to detect a bleeding portion in a body based on a detection result of an event detection unit (for example, the EVS 21) including a plurality of first pixels (for example, the pixel 302), each of the first pixels receiving light reflected in the body irradiated by a light source 30, and the event detection unit detecting, for each of the first pixels, that a luminance change amount due to the light incident on the first pixel exceeds a predetermined threshold value depending on a change in a wavelength of the light source 30. As a result, it is possible to detect a luminance change accompanying a wavelength change of the light source 30, and it is possible to detect a bleeding portion in the body of a patient or the like in an environment in which surgery is performed on the body of a patient or the like.

Furthermore, the light source control unit 43 that controls the light source 30 so as to change the wavelength of the light source 30 may be provided. As a result, the wavelength of the light source 30 can be reliably changed, so that the luminance change accompanying the wavelength change of the light source 30 can be reliably detected.

Furthermore, the light source control unit 43 may control the light source 30 so as to limit the wavelength of the light source 30 within a predetermined range. As a result, since a range in which the wavelength of the light source 30 is changed is limited, the luminance change accompanying the wavelength change of the light source 30 can be efficiently detected.

Furthermore, the predetermined range of the wavelength of the light source 30 may be set based on an absorption characteristic of blood depending on the change in the wavelength. As a result, it is possible to reliably detect a bleeding portion in the body of a patient or the like.

Furthermore, the bleeding detection unit may detect a position and a region of a bleeding portion in the body of a patient or the like. As a result, an operator such as an operating surgeon can reliably grasp a bleeding portion in the body of a patient or the like.

In addition, the bleeding detection unit may compare a waveform of the luminance change amount depending on the change in the wavelength of the light source 30 with a waveform of the absorption characteristic of the blood depending on the change in the wavelength. As a result, it is possible to reliably detect a bleeding portion in the body of a patient or the like.

Furthermore, the image processing unit 42 that generates an image in the body including the bleeding portion may be provided. As a result, an operator such as an operating surgeon can accurately grasp a bleeding portion in the body of a patient or the like.

Furthermore, the image in the body including the bleeding portion may include a figure (for example, the figure A2) indicating a position and a region of the bleeding portion in the body of the patient or the like. As a result, an operator such as an operating surgeon can accurately and reliably grasp a bleeding portion in the body of a patient or the like.

In addition, the image in the body including the bleeding portion may include an image (for example, text A3) indicating that there is the bleeding portion in the body. As a result, an operator such as an operating surgeon can reliably grasp that there is a bleeding portion in the body of a patient or the like.

Furthermore, in addition to the bleeding detection unit, the light source 30 and the event detection unit (for example, the EVS 21) may be provided to construct the medical observation system 10. With the medical observation system 10, as described above, it is possible to detect a bleeding portion in the body of a patient or the like in an environment in which surgery is performed on the body of the patient or the like.

Furthermore, there may be provided an image detection unit (for example, the RGB sensor 22) including a plurality of second pixels, each of the second pixels receiving light reflected in a body irradiated by the light source 30, and the image detection unit detecting an image in the body based on the light incident on each of the second pixels. As a result, it is possible to acquire an image in the body while detecting a bleeding portion in the body of a patient or the like.

In addition, the display device 50 that displays the bleeding portion while superimposing the bleeding portion on the image in the body may be provided. As a result, an operator such as an operating surgeon can grasp a positional relationship and a size relationship between the image in the body and the bleeding portion.

Furthermore, the display device 50 may display a position and a region of the bleeding portion on the image in the body. As a result, an operator such as an operating surgeon can accurately and reliably grasp the bleeding portion on the image in the body.

In addition, the inside of the body may be inside an abdominal cavity of a living body. That is, even when a surgical environment is an environment in which surgery is performed on the abdominal cavity of a living body such as a patient, a bleeding portion in the abdominal cavity can be detected.

### 2. Second Embodiment

### <2-1. Processing Example of Bleeding Detection>

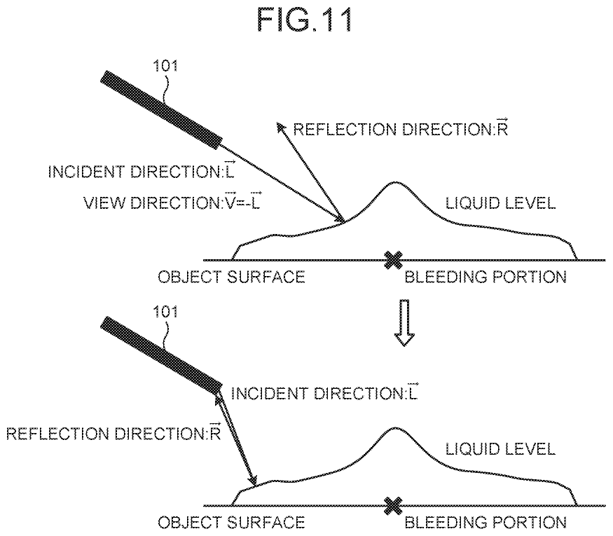

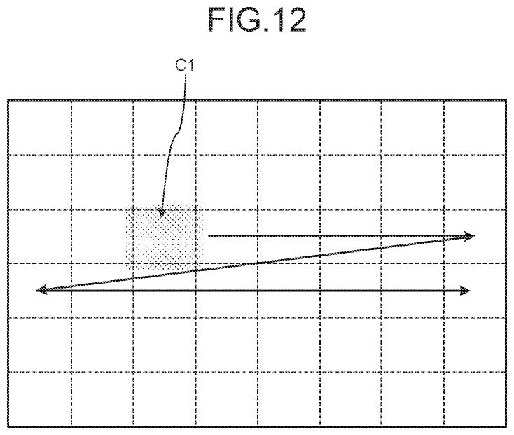

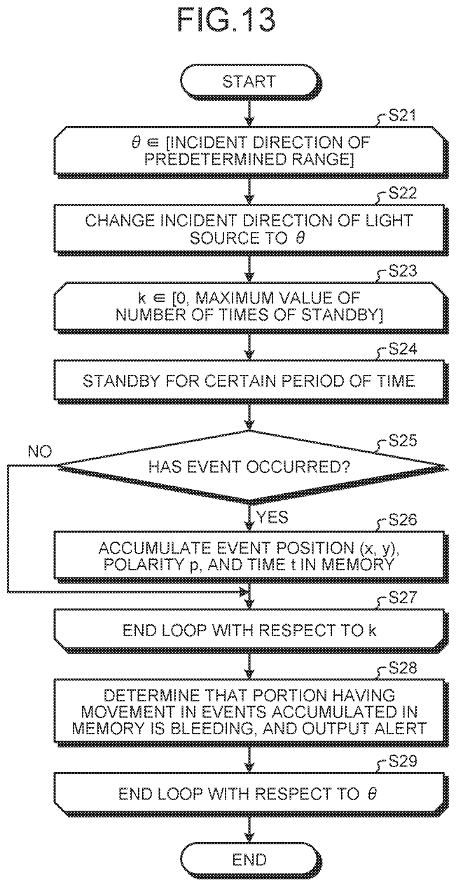

A processing example of bleeding detection by an irradiation direction change of irradiation light according to the present embodiment will be described with reference to FIGS. 11 to 13. FIG. 11 is a diagram illustrating an example of the bleeding detection processing by the irradiation direction change of the irradiation light according to the present embodiment. FIG. 12 is a diagram illustrating the irradiation direction change of the irradiation light according to the present embodiment. FIG. 13 is a flowchart illustrating an example of a flow of the bleeding detection processing by the irradiation direction change of the irradiation light according to the present embodiment.

As illustrated in FIG. 11, irradiation light guided by the optical system 101 travels toward an object surface corresponding to a surface of a body (for example, intraperitoneal) of a living body, and the irradiation light is reflected by the object surface or a liquid level of a bleeding portion, similarly to the first embodiment. A part of the reflected light (reflected light) is incident on the optical system 101. The light (incident light) incident on the optical system 101 is guided to the camera 20 (refer to FIG. 2) by the optical system 101. In the camera 20, an event is detected by the EVS 21 depending on a luminance change based on the incident light, and an image based on the incident light is obtained by the RGB sensor 22.

Here, a wavelength of the light source 30 is set to a wavelength at which light is well reflected from blood. When an incident direction of the light is known, a direction in which specular reflection of light occurs can be determined by a normal direction of an object hit by the light. Since there is no main light source that supplies light other than the light supplied from the light source 30 in the abdominal cavity, the present disclosure is limited to incident light from the light source 30 and secondary reflection thereof.

As illustrated in FIG. 12, by roughly adjusting an irradiation direction of the light source 30, that is, the incident direction with respect to the object surface or the liquid level so as to allow the irradiation direction to enter a small region C1 obtained by dividing an image, it is possible not only to reduce the influence of secondary reflection but also to focus on reflection of an object in a range of the small region C1. In practice, since a range in which an object is hit by light is determined depending on a distance from the optical system 101 of the camera 20 to the object hit by light, the range is determined depending on an intraperitoneal environment. However, it is preferable to change the irradiation direction of the light source 30 so as to cover the entire image. For example, the irradiation direction of the light source 30 is changed so as to scan all the small regions C1.

Since the EVS 21 is a sensor in which an event occurs only at a portion where a luminance change occurs, it is possible to efficiently perform processing by narrowing down a portion at which the luminance change occurs. In addition, since the EVS 21 can perform detection at high speed and high sensitivity, it is possible to change the irradiation direction (an incident direction with respect to an object) of the light source 30 in a short time. By using such an EVS 21, it is possible to detect only a luminance change accompanying an irradiation direction change of the light source 30 with high speed and high sensitivity, and it is possible to reliably detect a bleeding portion. That is, when the irradiation direction of the light source 30 is changed, specular reflection of light occurs according to a normal direction of an object hit by light. By detecting a change in the reflected light and detecting a shape of the object (for example, a change in wavefront shape of blood, and the like), a bleeding portion can be detected.

As illustrated in FIG. 13, as the bleeding detection processing, in step S21, the irradiation direction of the light source 30, that is, the incident direction (an incident angle θ) with respect to the object is changed. In the example of FIG. 13, steps S21 to S29 are performed in a loop, and the processing in the loop is repeatedly performed until the incident direction reaches a maximum value from a minimum value within a predetermined range. An increase amount of an incident angle θ for each loop is set to a predetermined value in advance.

In step S22, the incident direction of the light source 30 is changed by the light source control unit 43, and in step S23, k is changed from 0 to a maximum value of the number of times of standby. In the example of FIG. 13, steps S23 to S27 are performed in a loop, and the processing in the loop is repeatedly performed until k reaches the maximum value of the number of times of standby from 0.

Next, in step S24, the standby for a certain period of time is executed. The certain period of time is, for example, 0.01 seconds or the like, and is a period of time for event detection. An event is detected pixel by pixel by the EVS 21. It is noted that a threshold value related to occurrence of an event is set to a value with which a bleeding portion can be detected.

In step S25, the EVS 21 determines whether an event has occurred. When it is determined that the event has occurred (Yes in step S25), an event position (x, y), a polarity p, and a wavelength λ are associated with each other and accumulated in a memory in step S26.

After the processing in step S26 or when it is determined in step S25 that no event has occurred (No in step S25), the processing proceeds to step S27. When it is determined in step S27 that the loop has ended (k reaches the maximum value of the number of times of standby), it is determined in step S28 that bleeding has occurred at a portion having movement in the events accumulated in the memory, and an alert is output. This determination processing and output processing are executed by the signal processing unit 41. In step S29, when the loop is ended (when θ reaches the maximum value), the processing is completed.

According to such bleeding detection processing, one irradiation direction (the incident direction with respect to the object) of the light source 30 is determined. Within this range, standby is performed a certain number of times. Since light is reflected by blood, a luminance value increases as a normal direction is closer to a camera direction (an incident direction). When a blood liquid level moves within a standby period, the normal direction changes, and as such an intensity of specular reflection light changes and the luminance value changes. By repeating event detection by changing the irradiation direction of the light source 30 and aggregating the events, it is possible to know whether the liquid level is moving. In the case of bleeding, since there is movement in the liquid level, a portion having movement can be determined as a bleeding portion.

### <2-2. Action and Effect>

As described above, according to the second embodiment, the same effects as those of the first embodiment can be obtained. That is, there is provided a bleeding detection unit (for example, the signal processing unit 41) that detects a bleeding portion in a body based on a detection result of an event detection unit (for example, the EVS 21) configured to detect that a luminance change amount by light incident on a first pixel exceeds a predetermined threshold value for each of the first pixels depending on a change in an irradiation direction of the light source 30. As a result, it is possible to detect a luminance change accompanying a change in the irradiation direction of the light source 30, and it is possible to detect a bleeding portion in the body of a patient or the like in an environment in which surgery is performed on the body of the patient or the like.

Furthermore, the light source control unit 43 that controls the light source 30 so as to change the irradiation direction of the light source 30 may be provided. As a result, it is possible to reliably change the irradiation direction of the light source 30, and as such, it is possible to reliably detect the luminance change accompanying the change in the irradiation direction of the light source 30.

Furthermore, the light source control unit 43 may control the light source 30 so as to limit the irradiation direction of the light source 30 within a predetermined range. As a result, since a range in which the irradiation direction of the light source 30 is changed is limited, the luminance change accompanying the change in the irradiation direction of the light source 30 can be efficiently detected.

In addition, the bleeding detection unit may detect movement of blood related to a bleeding portion. As a result, it is possible to reliably detect a bleeding portion in the body of a patient or the like.

Furthermore, the bleeding detection unit may detect that the luminance change amount depending on the change in the irradiation direction of the light source 30 changes with the lapse of time. As a result, it is possible to accurately and reliably detect a bleeding portion in the body of a patient or the like.

### 3. Other Embodiments

The processing according to the above-described embodiments (or modifications) may be performed in various different modes (modifications) other than the above-described embodiments. For example, among the various types of processing described in the above embodiments, all or a part of the processing described as being automatically performed can be manually performed, or all or a part of the processing described as being manually performed can be automatically performed by a known method. In addition, the processing procedure, specific name, and information including various data and parameters illustrated in the document and the drawings can be freely and selectively changed unless otherwise specified. For example, the various types of information illustrated in each drawing are not limited to the illustrated information.

In addition, each component of each device illustrated in the drawings is functionally conceptual, and is not necessarily physically configured as illustrated in the drawings. That is, a specific form of distribution and integration of each device is not limited to the illustrated form, and all or a part thereof can be functionally or physically distributed and integrated in any unit depending on various loads, usage conditions, and the like.

In addition, the above-described embodiments (or modifications) can be appropriately combined within a range that does not contradict processing contents. Furthermore, the effects described in the present specification are merely examples and are not limited, and other effects may be obtained.

### 4. Application Example

The technology according to the present disclosure can be applied to a medical imaging system. The medical imaging system is a medical system using an imaging technology, and is, for example, an endoscope system or a microscope system. In the medical observation system 10 according to the present disclosure, the camera 20 can be applied to an endoscope 5001 or a microscope device 5301, the light source 30 can be applied to a light source device 5043, the processing apparatus 40 can be applied to a CCU 5039, and the display device 50 can be applied to a display device 5041. It is noted that, in the following, a basic operation and processing will be described with respect to an endoscope system and a microscope system, but actually, the operation and processing according to each embodiment can be executed. For example, a camera 5005 includes the EVS 21 and the RGB sensor 22. However, since the operation and processing of the EVS 21 and the RGB sensor 22 have been described in each embodiment, descriptions thereof will be omitted below.

### [Endoscope System]

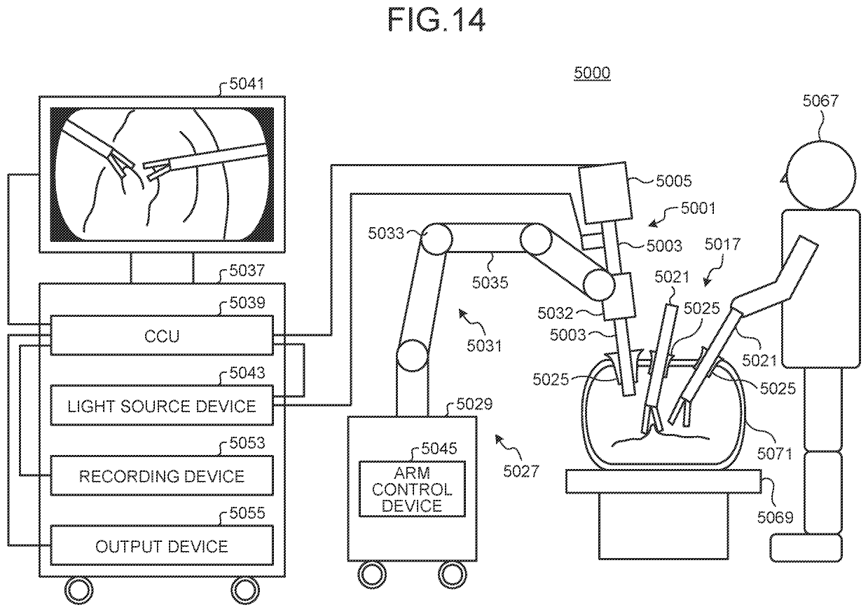

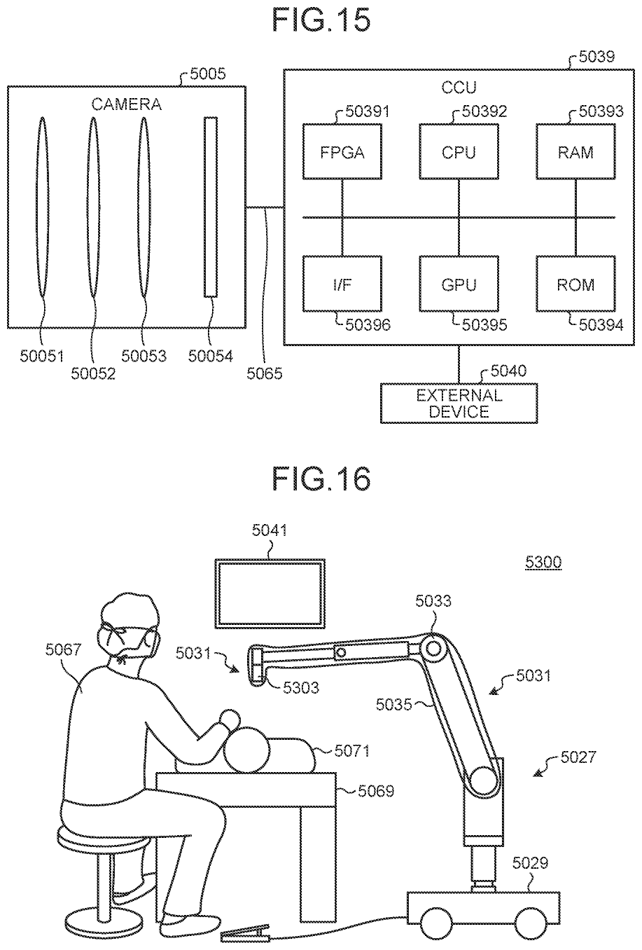

An example of the endoscope system will be described using FIGS. 14 and 15. FIG. 14 is a diagram illustrating an example of a schematic configuration of an endoscope system 5000 to which the technology according to the present disclosure is applicable. FIG. 15 is a diagram illustrating an example of a configuration of an endoscope 5001 and a camera control unit (CCU) 5039. FIG. 14 illustrates a situation where an operator (for example, a doctor) 5067 who is a participant of an operation performs the operation on a patient 5071 on a patient bed 5069 using the endoscope system 5000. As illustrated in FIG. 14, the endoscope system 5000 includes the endoscope 5001 that is a medical imaging device, the CCU 5039, a light source device 5043, a recording device 5053, an output device 5055, and a support device 5027 for supporting the endoscope 5001.

In endoscopic surgery, insertion assisting tools called trocars 5025 are punctured into the patient 5071. Then, a scope 5003 connected to the endoscope 5001 and surgical tools 5021 are inserted into a body of the patient 5071 through the trocars 5025. The surgical tools 5021 include: an energy device such as an electric scalpel; and forceps, for example.

A surgical image that is a medical image in which the inside of the body of the patient 5071 is captured by the endoscope 5001 is displayed on a display device 5041. The operator 5067 performs a procedure on a surgical target using the surgical tools 5021 while viewing the surgical image displayed on the display device 5041. The medical image is not limited to the surgical image, and may be a diagnostic image captured during diagnosis.

### [Endoscope]

The endoscope 5001 is an imaging section for capturing the inside of the body of the patient 5071, and is, for example, as illustrated in FIG. 15, a camera including a condensing optical system 50051 for condensing incident light, a zooming optical system 50052 capable of optical zooming by changing a focal length of the imaging section, a focusing optical system 50053 capable of focus adjustment by changing the focal length of the imaging section, and a light receiving sensor 50054. The endoscope 5001 condenses the light through the connected scope 5003 on the light receiving sensor 50054 to generate a pixel signal, and outputs the pixel signal through a transmission system to the CCU 5039. The scope 5003 is an insertion part that includes an objective lens at a distal end and guides the light from the connected light source device 5043 into the body of the patient 5071. The scope 5003 is, for example, a rigid scope for a rigid endoscope and a flexible scope for a flexible endoscope. The scope 5003 may be a direct viewing scope or an oblique viewing scope. The pixel signal only needs to be a signal based on a signal output from a pixel, and is, for example, a raw signal or an image signal. The transmission system connecting the endoscope 5001 to the CCU 5039 may include a memory, and the memory may store parameters related to the endoscope 5001 and the CCU 5039. The memory may be disposed at a connection portion of the transmission system or on a cable. For example, the memory of the transmission system may store the parameters before shipment of the endoscope 5001 or the parameters changed when current is applied, and an operation of the endoscope may be changed based on the parameters read from the memory. A set of the camera and the transmission system may be referred to as an endoscope. The light receiving sensor 50054 is a sensor for converting the received light into the pixel signal, and is, for example, a complementary metal-oxide-semiconductor (CMOS) imaging sensor. The light receiving sensor 50054 is preferably an imaging sensor having a Bayer array capable of color imaging. The light receiving sensor 50054 is also preferably an imaging sensor having a number of pixels corresponding to a resolution of, for example, 4K (3840 horizontal pixels×2160 vertical pixels), 8K (7680 horizontal pixels×4320 vertical pixels), or square 4K (3840 or more horizontal pixels×3840 or more vertical pixels). The light receiving sensor 50054 may be one sensor chip, or a plurality of sensor chips. For example, a prism may be provided to separate the incident light into predetermined wavelength bands, and the wavelength bands may be imaged by different light receiving sensors. A plurality of light receiving sensors may be provided for stereoscopic viewing. The light receiving sensor 50054 may be a sensor having a chip structure including an arithmetic processing circuit for image processing, or may be a sensor for time of flight (ToF). The transmission system is, for example, an optical fiber cable system or a wireless transmission system. The wireless transmission only needs to be capable of transmitting the pixel signal generated by the endoscope 5001, and, for example, the endoscope 5001 may be wirelessly connected to the CCU 5039, or the endoscope 5001 may be connected to the CCU 5039 via a base station in an operating room. At this time, the endoscope 5001 may transmit not only the pixel signal, but also simultaneously information (for example, a processing priority of the pixel signal and/or a synchronization signal) related to the pixel signal. In the endoscope, the scope may be integrated with the camera, and the light receiving sensor may be provided at the distal end of the scope.

### [Camera Control Unit (CCU)]

The CCU 5039 is a control device for controlling the endoscope 5001 and the light source device 5043 connected to the CCU 5039 in an integrated manner, and is, for example, as illustrated in FIG. 15, an image processing device including a field-programmable gate array (FPGA) 50391, a central processing unit (CPU) 50392, a random access memory 50393, a read-only memory (ROM) 50394, a graphics processing unit (GPU) 50395, and an interface (I/F) 50396. The CCU 5039 may control the display device 5041, the recording device 5053, and the output device 5055 connected to the CCU 5039 in an integrated manner. The CCU 5039 controls, for example, irradiation timing, irradiation intensity, and a type of an irradiation light source of the light source device 5043. The CCU 5039 also performs image processing, such as development processing (for example, demosaic processing) and correction processing, on the pixel signal output from the endoscope 5001, and outputs the processed image signal (for example, an image) to an external device such as the display device 5041. The CCU 5039 also transmits a control signal to the endoscope 5001 to control driving of the endoscope 5001. The control signal is information on an imaging condition such as a magnification or the focal length of the imaging section. The CCU 5039 may have a function to down-convert the image, and may be configured to be capable of simultaneously outputting a higher-resolution (for example, 4K) image to the display device 5041 and a lower-resolution (for example, high-definition (HD)) image to the recording device 5053.

The CCU 5039 may be connected to external equipment (such as a recording device, a display device, an output device, and a support device) via an IP converter for converting the signal into a predetermined communication protocol (such as the Internet Protocol (IP)). The connection between the IP converter and the external equipment may be established using a wired network, or a part or the whole of the network may be established using a wireless network. For example, the IP converter on the CCU 5039 side may have a wireless communication function, and may transmit the received image to an IP switcher or an output side IP converter via a wireless communication network, such as the fifth-generation mobile communication system (5G) or the sixth-generation mobile communication system (6G).

### [Light Source Device]

The light source device 5043 is a device capable of emitting the light having predetermined wavelength bands, and includes, for example, a plurality of light sources and a light source optical system for guiding the light of the light sources. The light sources are, for example, xenon lamps, light-emitting diode (LED) light sources, or laser diode (LD) light sources. The light source device 5043 includes, for example, the LED light sources corresponding to three respective primary colors of red (R), green (G), and blue (B), and controls output intensity and output timing of each of the light sources to emit white light. The light source device 5043 may include a light source capable of emitting special light used for special light observation, in addition to the light sources for emitting normal light for normal light observation. The special light is light having a predetermined wavelength band different from that of the normal light being light for the normal light observation, and is, for example, near-infrared light (light having a wavelength of 760 nm or longer), infrared light, blue light, or ultraviolet light. The normal light is, for example, the white light or green light. In narrow band imaging that is a kind of special light observation, blue light and green light are alternately emitted, and thus the narrow band imaging can image a predetermined tissue such as a blood vessel in a mucosal surface at high contrast using wavelength dependence of light absorption in the tissue of the body. In fluorescence observation that is a kind of special light observation, excitation light is emitted for exciting an agent injected into the tissue of the body, and fluorescence emitted by the tissue of the body or the agent as a label is received to obtain a fluorescent image, and thus the fluorescence observation can facilitate the operator to view, for example, the tissue of the body that is difficult to be viewed by the operator with the normal light. For example, in fluorescence observation using the infrared light, the infrared light having an excitation wavelength band is emitted to an agent, such as indocyanine green (ICG), injected into the tissue of the body, and the fluorescence light from the agent is received, whereby the fluorescence observation can facilitate viewing of a structure and an affected part of the tissue of the body. In the fluorescence observation, an agent (such as 5-aminolevulinic acid (5-ALA)) may be used that emits fluorescence in a red wavelength band by being excited by the special light in a blue wavelength band. The type of the irradiation light of the light source device 5043 is set by control of the CCU 5039. The CCU 5039 may have a mode of controlling the light source device 5043 and the endoscope 5001 to alternately perform the normal light observation and the special light observation. At this time, information based on a pixel signal obtained by the special light observation is preferably superimposed on a pixel signal obtained by the normal light observation. The special light observation may be an infrared light observation to observe a site inside the surface of an organ and a multi-spectrum observation utilizing hyperspectral spectroscopy. A photodynamic therapy may be incorporated.

### [Recording Device]

The recording device 5053 is a device for recording the pixel signal (for example, an image) acquired from the CCU 5039, and is, for example, a recorder. The recording device 5053 records an image acquired from the CCU 5039 in a hard disk drive (HDD), a Super Density Disc (SDD), and/or an optical disc. The recording device 5053 may be connected to a network in a hospital to be accessible from equipment outside the operating room. The recording device 5053 may have a down-convert function or an up-convert function.

### [Display Device]

The display device 5041 is a device capable of displaying the image, and is, for example, a display monitor. The display device 5041 displays a display image based on the pixel signal acquired from the CCU 5039. The display device 5041 may include a camera and a microphone to function as an input device that allows instruction input through gaze recognition, voice recognition, and gesture.

### [Output Device]

The output device 5055 is a device for outputting the information acquired from the CCU 5039, and is, for example, a printer. The output device 5055 prints, for example, a print image based on the pixel signal acquired from the CCU 5039 on a sheet of paper.

### [Support Device]

The support device 5027 is an articulated arm including a base 5029 including an arm control device 5045, an arm 5031 extending from the base 5029, and a holding part 5032 mounted at a distal end of the arm 5031. The arm control device 5045 includes a processor such as a CPU, and operates according to a predetermined computer program to control driving of the arm 5031. The support device 5027 uses the arm control device 5045 to control parameters including, for example, lengths of links 5035 constituting the arm 5031 and rotation angles and torque of joints 5033 so as to control, for example, the position and attitude of the endoscope 5001 held by the holding part 5032. This control can change the position or attitude of the endoscope 5001 to a desired position or attitude, makes it possible to insert the scope 5003 into the patient 5071, and can change the observed area in the body. The support device 5027 functions as an endoscope support arm for supporting the endoscope 5001 during the operation. Thus, the support device 5027 can play a role of a scopist who is an assistant holding the endoscope 5001. The support device 5027 may be a device for holding a microscope device 5301 to be described later, and can be called a medical support arm. The support device 5027 may be controlled using an autonomous control method by the arm control device 5045, or may be controlled using a control method in which the arm control device 5045 performs the control based on input of a user. The control method may be, for example, a master-slave method in which the support device 5027 serving as a slave device (replica device) that is a patient cart is controlled based on a movement of a master device (primary device) that is an operator console at a hand of the user. The support device 5027 may be remotely controllable from outside the operating room.

The example of the endoscope system 5000 to which the technology according to the present disclosure is applicable has been described above. For example, the technology according to the present disclosure may be applied to a microscope system.

### [Microscope System]

FIG. 16 is a diagram illustrating an example of a schematic configuration of a microscopic surgery system to which the technology according to the present disclosure is applicable. In the following description, the same components as those of the endoscope system 5000 will be denoted by the same reference numerals, and the description thereof will not be repeated.

FIG. 16 schematically illustrates a situation where the operator 5067 performs an operation on the patient 5071 on the patient bed 5069 using a microscopic surgery system 5300. For the sake of simplicity, FIG. 16 does not illustrate a cart 5037 among the components of the microscopic surgery system 5300, and illustrates the microscope device 5301 instead of the endoscope 5001 in a simplified manner. The microscope device 5301 may refer to a microscope 5303 provided at the distal end of the links 5035, or may refer to the overall configuration including the microscope 5303 and the support device 5027.

As illustrated in FIG. 16, during the operation, the microscopic surgery system 5300 is used to display an image of a surgical site captured by the microscope device 5301 in a magnified manner on the display device 5041 installed in the operating room. The display device 5041 is installed in a position facing the operator 5067, and the operator 5067 performs various procedures, such as excision of an affected part, on the surgical site while observing the state of the surgical site using the image displayed on the display device 5041. The microscopic surgery system is used in, for example, ophthalmic operation and neurosurgical operation.

The respective examples of the endoscope system 5000 and the microscopic surgery system 5300 to which the technology according to the present disclosure is applicable have been described above. Systems to which the technology according to the present disclosure is applicable are not limited to such examples. For example, the support device 5027 can support, at the distal end thereof, another observation device or another surgical tool instead of the endoscope 5001 or the microscope 5303. Examples of the other applicable observation device include forceps, tweezers, a pneumoperitoneum tube for pneumoperitoneum, and an energy treatment tool for incising a tissue or sealing a blood vessel by cauterization. By using the support device to support the observation device or the surgical tool described above, the position thereof can be more stably fixed and the load of the medical staff can be lower than in a case where the medical staff manually supports the observation device or the surgical tool. The technology according to the present disclosure may be applied to a support device for supporting such a component other than the microscope.

The technology according to the present disclosure can be suitably applied to the endoscope 5001, the microscope device 5301, the CCU 5039, the display device 5041, the light source device 5043, and the like among the above-described configurations. Specifically, the operation and processing according to each embodiment can be executed in the endoscope system 5000, the microscopic surgery system 5300, and the like. By applying the technology according to the present disclosure to the endoscope system 5000, the microscopic surgery system 5300, and the like, it is possible to detect a bleeding portion in the body.

### 5. Appendix

It is noted that the present technology can also have the following configurations.

(1)

A medical information processing apparatus comprising:
a bleeding detection unit configured to detect a bleeding portion in a body based on a detection result of an event detection unit including a plurality of first pixels, each of the first pixels receiving light reflected in the body irradiated by a light source, the event detection unit detecting, for each of the first pixels, that a luminance change amount due to the light incident on the first pixel exceeds a predetermined threshold value depending on a change in a wavelength or an irradiation direction of the light source.

(2)

The medical information processing apparatus according to (1), further comprising:
a light source control unit configured to control the light source so as to change the wavelength of the light source.

(3)

The medical information processing apparatus according to (2), wherein
the light source control unit controls the light source so as to limit the wavelength of the light source within a predetermined range.

(4)

The medical information processing apparatus according to (3), wherein
the predetermined range is set based on an absorption characteristic of blood depending on the change in the wavelength.

(5)

The medical information processing apparatus according to (1), further comprising:
a light source control unit configured to control the light source so as to change the irradiation direction of the light source.

(6)

The medical information processing apparatus according to (5), wherein
the light source control unit controls the light source so as to limit the irradiation direction of the light source within a predetermined range.

(7)

The medical information processing apparatus according to any one of (1) to (6), wherein
the bleeding detection unit detects a position and a region of the bleeding portion.

(8)

The medical information processing apparatus according to any one of (1) to (7), wherein
the bleeding detection unit detects movement of blood related to the bleeding portion.

(9)

The medical information processing apparatus according to any one of (1) to (8), wherein
the bleeding detection unit compares a waveform of the luminance change amount depending on the change in the wavelength with a waveform of an absorption characteristic of blood depending on the change in the wavelength.

(10)

The medical information processing apparatus according to any one of (1), (5) to (8), wherein
the bleeding detection unit detects that the luminance change amount depending on the change in the irradiation direction changes with lapse of time.

(11)

The medical information processing apparatus according to any one of (1) to (10), further comprising:
an image processing unit configured to generate an image in the body including the bleeding portion.

(12)

The medical information processing apparatus according to (11), wherein
the image includes a figure indicating a position and a region of the bleeding portion.

(13)

The medical information processing apparatus according to (11), wherein
the image includes an image indicating that the bleeding portion is present in the body.

(14)

A medical observation system comprising:
a light source;
an event detection unit including a plurality of first pixels, each of the first pixels receiving light reflected in a body irradiated by the light source, the event detection unit being configured to detect, for each of the first pixels, that a luminance change amount due to the light incident on the first pixel exceeds a predetermined threshold value depending on a change in a wavelength or an irradiation direction of the light source; and
a bleeding detection unit configured to detect a bleeding portion in the body based on a detection result of the event detection unit.

(15)

The medical observation system according to (14), further comprising:
a light source control unit configured to control the light source so as to change the wavelength of the light source.

(16)

The medical observation system according to (14), further comprising:
a light source control unit configured to control the light source so as to change the irradiation direction of the light source.

(17)

The medical observation system according to any one of (14) to (16), further comprising:
an image detection unit including a plurality of second pixels, each of the second pixels receiving the light reflected in the body irradiated by the light source, the image detection unit being configured to detect an image in the body based on the light incident on each of the plurality of second pixels.

(18)

The medical observation system according to (14), further comprising:
a display device configured to display the bleeding portion while superimposing the bleeding portion on an image in the body.

(19)

The medical observation system according to (15), wherein
the display device displays a position and a region of the bleeding portion on the image in the body.

(20)

A medical information processing method comprising:
by a medical information processing apparatus,
detecting a bleeding portion in a body based on a detection result of an event detection unit including a plurality of first pixels, each of the first pixels receiving light reflected in the body irradiated by a light source, the event detection unit detecting, for each of the first pixels, that a luminance change amount due to the light incident on the first pixel exceeds a predetermined threshold value depending on a change in a wavelength or an irradiation direction of the light source.

(21)

A medical observation system including the medical information processing apparatus according to any one of (1) to (13).

(22)

A medical information processing method including a step performed by the medical information processing apparatus according to any one of (1) to (13).

### REFERENCE SIGNS LIST

10 MEDICAL OBSERVATION SYSTEM
20 CAMERA
21 EVS
22 RGB SENSOR
23 BEAM SPLITTER
30 LIGHT SOURCE
40 PROCESSING APPARATUS
41 SIGNAL PROCESSING UNIT
42 IMAGE PROCESSING UNIT
43 LIGHT SOURCE CONTROL UNIT
44 CONTROL UNIT
50 DISPLAY DEVICE
100 CAMERA HEAD
101 OPTICAL SYSTEM
210 PIXEL ARRAY UNIT
211 DRIVING CIRCUIT
212 ARBITER UNIT
213 COLUMN PROCESSING UNIT
214 SIGNAL PROCESSING UNIT
300 PIXEL ARRAY UNIT
302 PIXEL
304 LIGHT RECEIVING UNIT
306 PIXEL SIGNAL GENERATION UNIT
308 DETECTION UNIT
A1 BLEEDING PORTION
A2 FIGURE
A3 TEXT
B1 IMAGE
B2 ORGAN
C1 SMALL REGION

1. A medical information processing apparatus comprising:
a bleeding detection unit configured to detect a bleeding portion in a body based on a detection result of an event detection unit including a plurality of first pixels, each of the first pixels receiving light reflected in the body irradiated by a light source, the event detection unit detecting, for each of the first pixels, that a luminance change amount due to the light incident on the first pixel exceeds a predetermined threshold value depending on a change in a wavelength or an irradiation direction of the light source.

2. The medical information processing apparatus according to claim 1, further comprising:
a light source control unit configured to control the light source so as to change the wavelength of the light source.

3. The medical information processing apparatus according to claim 2, wherein
the light source control unit controls the light source so as to limit the wavelength of the light source within a predetermined range.

4. The medical information processing apparatus according to claim 3, wherein
the predetermined range is set based on an absorption characteristic of blood depending on the change in the wavelength.

5. The medical information processing apparatus according to claim 1, further comprising:
a light source control unit configured to control the light source so as to change the irradiation direction of the light source.

6. The medical information processing apparatus according to claim 5, wherein
the light source control unit controls the light source so as to limit the irradiation direction of the light source within a predetermined range.

7. The medical information processing apparatus according to claim 1, wherein
the bleeding detection unit detects a position and a region of the bleeding portion.

8. The medical information processing apparatus according to claim 1, wherein
the bleeding detection unit detects movement of blood related to the bleeding portion.

9. The medical information processing apparatus according to claim 1, wherein
the bleeding detection unit compares a waveform of the luminance change amount depending on the change in the wavelength with a waveform of an absorption characteristic of blood depending on the change in the wavelength.

10. The medical information processing apparatus according to claim 1, wherein
the bleeding detection unit detects that the luminance change amount depending on the change in the irradiation direction changes with lapse of time.

11. The medical information processing apparatus according to claim 1, further comprising:
an image processing unit configured to generate an image in the body including the bleeding portion.

12. The medical information processing apparatus according to claim 11, wherein
the image includes a figure indicating a position and a region of the bleeding portion.

13. The medical information processing apparatus according to claim 11, wherein
the image includes an image indicating that the bleeding portion is present in the body.

14. A medical observation system comprising:
a light source;
an event detection unit including a plurality of first pixels, each of the first pixels receiving light reflected in a body irradiated by the light source, the event detection unit being configured to detect, for each of the first pixels, that a luminance change amount due to the light incident on the first pixel exceeds a predetermined threshold value depending on a change in a wavelength or an irradiation direction of the light source; and
a bleeding detection unit configured to detect a bleeding portion in the body based on a detection result of the event detection unit.

15. The medical observation system according to claim 14, further comprising:
a light source control unit configured to control the light source so as to change the wavelength of the light source.

16. The medical observation system according to claim 14, further comprising:
a light source control unit configured to control the light source so as to change the irradiation direction of the light source.

17. The medical observation system according to claim 14, further comprising:
an image detection unit including a plurality of second pixels, each of the second pixels receiving the light reflected in the body irradiated by the light source, the image detection unit being configured to detect an image in the body based on the light incident on each of the plurality of second pixels.

18. The medical observation system according to claim 14, further comprising:
a display device configured to display the bleeding portion while superimposing the bleeding portion on an image in the body.

19. The medical observation system according to claim 18, wherein
the display device displays a position and a region of the bleeding portion on the image in the body.

20. A medical information processing method comprising:
by a medical information processing apparatus,
detecting a bleeding portion in a body based on a detection result of an event detection unit including a plurality of first pixels, each of the first pixels receiving light reflected in the body irradiated by a light source, the event detection unit detecting, for each of the first pixels, that a luminance change amount due to the light incident on the first pixel exceeds a predetermined threshold value depending on a change in a wavelength or an irradiation direction of the light source.
